(19) United States
(12) Patent Application Publication
(10) Pub. No.: US 2010/0222642 A1
(43) Pub. Date: Sep. 2, 2010

(54) EXPANDABLE DIGESTIVE PILL AND METHOD OF USE THEREOF

(75) Inventor: Karen I. Trovato, Putnam Valley, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/295,477

(22) PCT Filed: Mar. 19, 2007
(86) PCT No.: PCT/IB2007/050956

§ 371 Date: Sep. 30, 2008

Related U.S. Application Data
(60) Provisional application No. 60/787,454, filed on Mar. 30, 2006.

(51) Int. Cl. A61F 2/02

(52) U.S. Cl. 600/37

(57) ABSTRACT

A digestible capsule containing a balloon or expandable material is disclosed. The capsule comprises a container containing a gas therein and a means for generating a signal to the container to release the gas contained therein to expand the balloon to a known size or shape. In one aspect of the invention, a first portion of said capsule material is dissolved a predetermined time after encountering an acid, and the means for generating the signal generates the signal to the container after exposure to the acid. In another aspect of the invention, the means generates the signal to the container in response to an external signal. In still another aspect of the invention, the capsule further includes acid measuring means to determine an acid level and the signal generating means generates the signal in response to a known acid level. In still another aspect the capsule includes means for generating a second signal to cause the capsule to return to its original volume or less. In still another aspect the capsule material is dissolved a predetermined time after encountering an acid or allows for the leakage of the gas released therein.

This application is a continuation-in-part and claims priority to the earlier filing date of that patent application entitled, System And Method For Controlling Traversal Of An Ingested Capsule, filed in the US Patent Office on Jan. 18, 2005, and afforded patent application Ser. No. 60/644,518, the contents of which are incorporated by reference herein.

This invention is related to the field of electronically controlled capsule and more specifically to a non-surgical means for filling the stomach to encourage weight loss by creating a feeling of fullness.

Weight loss is an American preoccupation with approximately 44 percent of the population actively trying to lose weight. Unfortunately even more people should be losing weight as approximately 64 percent (129.6 million) of the American population are said to be overweight or obese. Americans are expected to spend in the order of $40 billion dollars on weight-control pills, gym memberships, diet plans and related foods in the quest to lose excessive weight. Obesity and excessive weight have been shown to increase the risk of developing type 2 diabetes, heart disease, some forms of cancer, and other disabling medical condition. The direct and indirect costs, including medical costs and lost productivity, were estimated at $117 billion dollars nationally for the year 2000, according to the 2001 Surgeon General's Call to Action to Prevent and Decrease Overweight and Obesity.

Numerous diet plans, theories, pills and devices are available to assist in losing excessive weight. Surgery, in many cases is used for overweight and obese people with medical risks such as diabetes where conventional weight-loss techniques fail.

Gastric banding, referred to as lap banding, is one procedure that is often used for controlling and forcing the loss of excessive weight. Gastric banding places a constricting band below the entry to the stomach and prohibits the entry of large food particles into the stomach. Thus, food is restricted to the upper, smaller, portion of the stomach above the band. The gastric band device is introduced through tiny incisions in the abdomen and is placed around the upper part of the stomach. The inclusion of the band around the stomach results in a pouch significantly reduced in size compared to the normal stomach and dramatically reduces the stomach's functionally capability. In this manner, a patient is made to feel full even though the greater part of the stomach remains empty. Over 150,000 of such bands have been placed worldwide. However, the procedure is very expensive; ranging from $10,000 in Mexico to between $17,000 & $30,000 in the United States. However, gastric banding has a disadvantage as the patient may often feel discomfort and there is a possibility of the weight returning when the band is removed. After Gastric Banding, patients cannot swallow a pill larger than an aspirin, making it impossible for some patients who may need aspirin or other orally delivered medication. Gastric bypass surgery is an even more radical surgery used for those persons who are obese. Gastric bypass, similar to gastric banding, reduces the stomach functional capacity by cutting off a significant portion of the stomach. Stomach stapling is a common method used to reduce the stomach size and further cause part of the small intestine to be bypassed. While gastric bypass is effective in causing the desired weight loss, it is a major surgery incurring the associated risks and costs. Stomach stapling is also not easily reversible.

As both gastric banding and gastric bypass require some form of surgery, these procedures carry clear risks from infection and anesthesia. Furthermore, medications often have side effects, which are sometimes only discovered after the larger population has taken the medications and accurate records become available.

Other, non-surgical, methods have been disclosed to reduce the intake capacity of the stomach. One example, U.S. Pat. No. 4,133,315, entitled “Method and Apparatus for Reducing Obesity,” to Berman discloses the introduction of a bag or balloon in a patient's stomach through a nose or mouth tube that is used to inflate or deflate the bag. Berman further discloses a step requiring alternately inflating the bag and having the patient eating orally by placing food in the mouth and swallowing. U.S. Pat. No. 5,993,473, entitled “Expandable Body Device for the Gastric Cavity and Method,” to Chan discloses the insertion of a device through the body cavity to create a balloon internal to the body. An external attachment is maintained to inflate or deflate the balloon.

However, these methods each present problems, as they require an external means to control the expansion or deflation of the balloon, which is either inconvenient and/or uncomfortable to implement. Hence, there is need in the industry for a minimally invasive means for filling the stomach cavity without requiring an external means for controlling the inflation or deflation.

A digestible capsule containing a balloon or expandable material is disclosed. The capsule comprises a container containing a gas therein and a means for generating a signal to the container to release the gas contained therein to expand the balloon to a known size or shape. In one aspect of the invention, a first portion of said capsule material is dissolved a predetermined time after encountering an acid, and the means for generating the signal generates the signal to the container after exposure to the acid. In another aspect of the invention, the means generates the signal to the container in response to an external signal. In still another aspect of the invention, the capsule further includes acid measuring means to determine an acid level and the signal generating means generates the signal in response to a known acid level. In still another aspect the capsule includes means for generating a second signal to cause the capsule to return to its original volume or less. In still another aspect the capsule material is dissolved a predetermined time after encountering an acid or allows for the leakage of the gas released therein.

The electronically controlled pill described herein contains compressed gas, which inflates a balloon upon arrival in the stomach, determined by pH, elapsed time, or external validation of arrival such as might be performed with an imaging system. This gives the stomach a full feeling by reducing available space for food. Once inflated, the pill can be deflated, either after a pre-determined period of time or by remote control. This makes the procedure testable, reversible and controllable. Another benefit is that by using several balloons, the stomach may be filled gradually and safely to any level deemed appropriate by a doctor.

More than one ‘Weight Loss ePill’ can be used in order to fill the stomach more completely.

Since the ePill carries the gas source with it, no external pressurizing link follows the device. The ePill is also surgery free, since it inflates upon arrival in the stomach, and deflates on schedule or upon RF command. There should be no nutritional deficiency that is not easily remedied by discontinuing the use of the pills. Finally, the size of objects eaten will not be restricted, however the total volume that is comfortable at one sitting will be reduced.

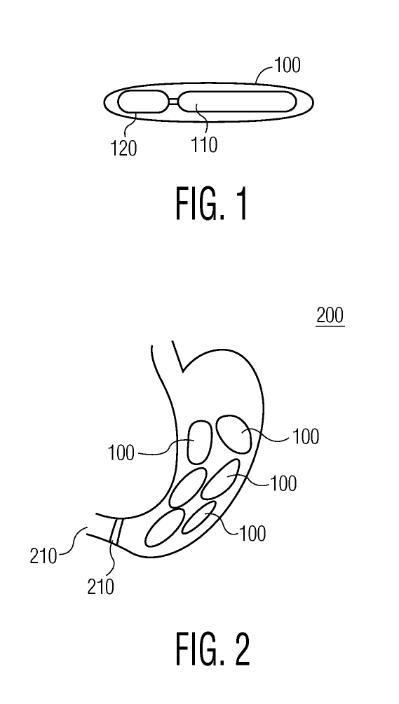

FIG. 1 illustrates a digestive pill in accordance with the principles of the invention;

FIG. 2 illustrates a plurality of digestive pills in accordance with the principles of the invention; and

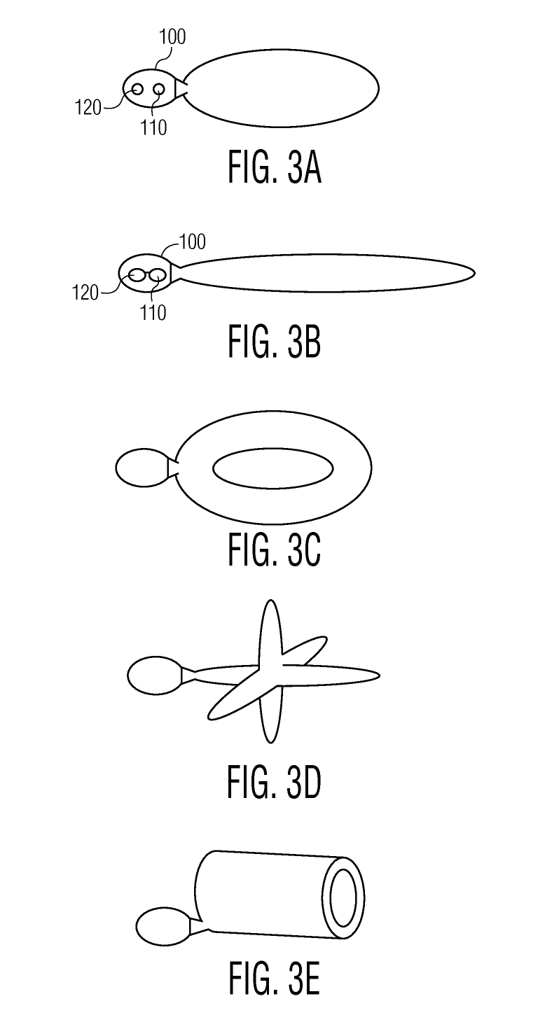

FIGS. 3(a)-3(e) illustrate several example shapes of balloons or expandable material that may be used in accordance with the principles of the invention.

It is to be understood that these drawings are solely for purposes of illustrating the concepts of the invention and are not intended as a definition of the limits of the invention. The embodiments shown in the figures herein and described in the accompanying detailed description are to be used as illustrative embodiments and should not be construed as the only manner of practicing the invention. Also, the same reference numerals, possibly supplemented with reference characters where appropriate, have been used to identify similar elements

The volume of the human stomach varies depending on the person. Generally, a human stomach has a volume of about one liter, but it can be distended up to four liters. The stomach typically contracts approximately 3 times per minute, pushing the contents within the stomach towards the pylorus. As the pyloric sphincter contracts, the mixture is pushed back into the body of the stomach. This mixture becomes reduced into chyme—a semi-fluid substance—within the stomach cavity and each minute 6 to 10 milliliters of chyme is emptied into the intestine. Therefore, with each contraction, no more than about 3 milliliters of chyme is conveyed through to the pylorus.

FIG. 1 illustrates a digestive capsule 100, referred to herein as an ePill, represented as a conventional pill or capsule made of a material that may be expanded to a predetermined size in accordance with the principles of the invention. In the illustrated embodiment shown in FIG. 1, the digestive ePill 100 is represented as a capsule that includes a gas capsule 110 containing up to about 0.125 cm³ of a gas. The gas, when released, can cause the ePill 100 to inflate or expand a known volume, preferably, at least 6.25 cm³ or 6.25 ml. Thus, as the pyloric sphincter opens to let only about 3 milliliters of chyme into the intestine, and ePill 100 expanded to a volume of about 6.25 ml has sufficient volume to avoid passage into the intestine.

Also illustrated is a means 120 in communication with gas capsule 110, which is used to control the release of gas from gas canister 110. In one embodiment of the invention, means 120 may consist of a computer processor and memory that includes computer instructions, code or software that may be executed in response to known events to trigger gas canister 110 to release the gas contained therein. For example, means 120 may further include an acid measurement device which when exposed to the acidic contents of the stomach determines when a known level of acidity (measured by a pH level) is achieved. For example the acidity level in the stomach is in the range of 1.4 and 4.0 while the pH level of the rest of the GI tract is typically above 4. Thus, taking food with the ePill disclosed herein will cause the stomach to release more hydrochloric acid (HCl), typically having a pH of about 1.4, which will in turn lower total stomach pH and ensure safe deployment.

In response to the acid measurement device determining a known level of acidity, the computer system generates a trigger signal to gas canister 110 to expand the capsule 100 or the attached balloon. In another embodiment of the invention, the acid determining device may generate a trigger signal directly to gas capsule 110. In still another aspect of the invention, the capsule 100 may include a section that is dissolved by the surrounding stomach acid, causing the electronic circuitry contained therein (i.e., means 120) to be activated. In this aspect of the invention, the dissolved section of capsule 100 may expose electrically isolated terminals connected to the means 120. When the electrically isolated terminals are exposed to an acidic solution, an electrical connection is made between the terminals that activate means 120 to generate a trigger signal to gas canister 110. In another embodiment, means 120 may be activated by a received signal provided by a transmitter outside the stomach. The received signal causes means 120 to generate a trigger signal that is provided to gas capsule 110. In this embodiment of the invention, means 120 may include an antenna and receiver responsive to a selected transmission frequency and a computer system to process the received signal. In still another embodiment of the invention, means 120 may be activated after a predetermined time period. In still another embodiment of the invention, capsule 100 may include a switch which when depressed causes means 120 to generate a signal to gas canister 110, after a predetermined time. For example, in one aspect of the invention when a switch is depressed, a timer, e.g., countup or countdown, is activated and responsive to the expiration of the timer, a trigger signal is provided to gas canister 110. In this case, the combination of the switch and timer constitute the computer system of means 120. As would be recognized, means 120 may also generate a signal to gas canister 110 based on one or more of the above referred-to criterion. Although the examples of means 120 have been discussed in detail herein it would be recognized that it is in the scope of the invention that the means 120 may be a general purpose processor in communication with a memory that executes computer code to implement the processing described herein. The general purpose processor may execute general computer software instructions. Similarly, means 120 may be a special purpose processor in communication with a memory that executes computer code to implement the processing described herein. The special purpose processor may execute special purpose computer software instructions. Furthermore, the computer system may be a processor, such as an ASIC (Application Specific Integrated Circuit) or a FPGA (Field Programmable Gate Array) that executes hardware specific instructions.

Although the ePill 100 has been explained with regard to a capsule 100 that is expandable, it would be recognized that it would be within the scope of the invention that only a portion of the capsule is expanded by the release gas contained within gas canister 110. For example, an inflatable balloon attached to end of capsule 100. In this case, the gas canister 110 releases the gas into the balloon portion of capsule 100 to expand the balloon to the required volume. Details of inflating a flexible balloon attached to capsule 100 is described in the aforementioned patent application Ser. No. ______ (ID 779006), and need not be discussed in detail herein.

In the present application, the capsule 100 may include a portion of flexible material or an attached flexible material (a balloon) and inflated in a manner described in the aforementioned patent application. These capsules may be made of non-absorbable material such as polyethylene or polypropylene so that they pass through the body. Balloons for the stomach are known. For example silicone elastomer material of the BioEnterics Intragastric Ballon (BIB) or polyethylene, polypropylene, PVC, PVCD, PET or Teflon may be used of the balloons and are well known in the art.

FIG. 2 illustrates an example of the use of a plurality of ePills 100 in a stomach cavity 200. In this illustrative example, a plurality of capsules fills the volume of the stomach 200 without causing distention. Each of the illustrated ePills 100 is expanded to a known volume, which is greater than a nominal pyloric sphincter 220 size that prevents the expanded ePill 100 from passing through the pyloric sphincter 210 to the pylorus 220.

In still another aspect of the invention, the capsules 100 may be deflated to a volume wherein capsules 100 may pass through pyloric sphincter 210 and be expelled from the stomach and the body. In one aspect of the invention, capsule 100 may be deflated after a predetermined time. For example, the released gas may escape capsule 100 over a known period through leakage into the stomach. In another example, the material of capsule 100 may dissolve after a predetermined time period leaving only the smaller gas capsule 110 and means 120 to be expelled. Dissolvable material, such as that used for dissolvable sutures, is known to last over two weeks in the stomach. Examples of dissolvable material include catgut (from the submucosa of sheep), which can be treated with chromic acid to last over 20 days, and a synthetic called Dexon (polyglycolic acid). These are well known in the art and need not be discussed herein.

In still another embodiment of the invention, means 120 may be used to deflate capsule 100. In this case, means 120 may provide a signal to cause the gas contained within ePill 100 to be released; returning ePill 100 to its original size. Means 120 may be activated to generate a deflating signal based on predetermined time period after an inflation signal or by the reception of an external signal. The aforementioned patent application Ser. No. ______(ID 779006), further describes means for deflating a flexible balloon attached to capsule 100 and need not be discussed in detail herein.

In alternative embodiments of the invention, non-round balloons may be used to fill the stomach more efficiently, since the non-round balloons will trap space between them more easily, and thus require fewer pills at one time. FIGS. 3(a) and (b) illustrate examples of balloons having a spherical and elongated shape. A preferred shape is shown in FIG. 3(c), which is a toroidal shape. The toroidal shape is advantageous as it allows food to pass through while maintaining a shape that is not likely to cause injury, yet occupying a larger external volume than the actual gas volume. FIGS. 3(d) and 3(e) illustrate further examples of balloons that may be implemented. FIG. 3(d) illustrates a three-dimensional star shape that may be used. In this case, when a plurality of such star shape balloons are ingested they may leave space to allow for the passage of liquids. Similarly, the cylindrical balloon shape in FIG. 3(e) allows liquids to passage through the balloon to avoid blockages of liquids in the stomach. In still another embodiment of the invention, when part of the stomach/GI tract is surgically removed, these controllable balloons may be used to stretch the stomach/GI tract gradually, so that its size is increased. The balloons may be used in conjunction with a means for detecting bleeding. At the proper location the balloon expands, creating direct pressure to stop the bleeding. This may occur in regions other than the stomach. In such cases, the balloon is a hollowed cylindrical shape to allow fluids to pass while maintaining the expanded shape. FIG. 3(e) shows such a configuration. The location of the ePill 100 with respect to the balloon does not have to be centered as is shown FIG. 3(e). In still another embodiment, the balloons may be inflated to a particular pressure, so that the overall pressure on the GI tract or stomach is not so much as to rupture the organ.

While there has been shown, described, and pointed out fundamental novel features of the present invention as applied to preferred embodiments thereof, it will be understood that various omissions and substitutions and changes in the apparatus described, in the form and details of the devices disclosed, and in their operation, may be made by those skilled in the art without departing from the spirit of the present invention. For example, although the present invention has been described with regard to spherical, elongated or oblong balloons, it would be recognized that other shapes that are easily digestible may also be used without altering the scope of the invention. For example, although the ePill 100 has been shown as a capsule, it would be within the scope of the invention that the cross-sectional shape of the balloon may be torus. In this case, fluids may pass through the center of capsule 100.

Accordingly, it is expressly intended that all combinations of those elements that perform substantially the same function in substantially the same way to achieve the same results are within the scope of the invention. Substitutions of elements from one described embodiment to another are also fully intended and contemplated.

What is claimed is:

1. A digestible capsule, a portion of which includes an expandable material, the capsule containing therein:
a container containing a gas; and
a means for generating a signal to said container to release the gas contained therein to expand the expandable material portion of the capsule to at least a known size.

2. The capsule as recited in claim 1, wherein a first portion of said capsule material is dissolved a predetermined time after encountering an acid.

3. The capsule as recited in claim 1, wherein said capsule material is dissolved a predetermined time after encountering an acid.

4. The capsule as recited in claim 2, wherein a second, remaining, portion of said capsule material is dissolved a second predetermined time after encountering said acid.

5. The capsule as recited in claim 4, wherein said means generates the signal to the container after exposure to the acid.

6. The capsule as recited in claim 1, wherein said means generates the signal to the container in response to an external signal.

7. The capsule as recited in claim 1, further comprising:
acid measuring device to determine an acid level.

8. The capsule as recited in claim 7, wherein said signal generating means generates the signal in response to a known acid level.

9. The capsule as recited in claim 1, further comprising:
means for generating a second signal to cause said capsule to return to its original volume or less.

10. The capsule as recited in claim 1, wherein the capsule material allows for the leakage of said gas released therein.

11. The capsule as recited in claim 1, wherein said known volume is at least 6.25 milliliters.

12. The capsule as recited in claim 1, further comprising a switch.

13. The capsule as recited in claim 1, further comprising a timer which is activated in response to a trigging signal.

14. A method for decreasing the volume intake of a stomach cavity, said mean comprising the step of:
ingesting at least one digestible pill, the pill comprising:
a covering material, a portion of which includes a flexible material;
a container containing a gas within the outer covering; and
a means for generating a signal to said container to release the gas contained therein to expand the flexible material to at least a known size.

15. The method as recited in claim 14, wherein a first portion of said covering material is dissolved a predetermined time after encountering an acid.

16. The method as recited in claim 14, wherein said covering material is dissolved a predetermined time after encountering an acid

17. The method as recited in claim 15, wherein a second, remaining, portion of said covering material is dissolved a second predetermined time after encountering said acid.

18. The method as recited in claim 17, wherein said means generates the signal to the container after exposure to the acid.

19. The method as recited in claim 14, wherein said means generates the signal to the container in response to an external signal.

20. The method as recited in claim 14, wherein said pill further comprising:
acid measuring means to determine an acid level.

21. The method as recited in claim 20, wherein said signal generating means generates the signal in response to a known acid level.

22. The method as recited in claim 14, wherein said pill further comprising:
means for generating a second signal to cause said capsule to return to its original volume or less.

23. The method as recited in claim 14, wherein the covering material allows for the leakage of said gas released therein.

24. The method as recited in claim 14, wherein said known volume is at least 6.25 milliliters.

25. The capsule as recited in claim 1, wherein the expandable portion is a balloon.

26. The capsule as recited in claim 25, wherein the balloon possess a shape selected from the group consisting of: spherical, elongated, star, toridal, and hollow cylindrical.

27. The capsule as recited in claim 26, wherein the cross-sectional shape of the balloon allows for the passage of fluids therethrough.

28. The capsule as recited in claim 1, wherein the expandable material is filled to contain a known gas pressure.
